(19) United States
(12) Patent Application Publication
(10) Pub. No.: US 2005/0064555 A1
(43) Pub. Date: Mar. 24, 2005

(54) Ciliary neurotrophic factor variants

(75) Inventors: Shannon Marshall, San Francisco, CA (US); Maria Barbosa, San Dimas, CA (US)

Correspondence Address: Robin M. Silva, Esq.;Dorsey & Whitney LLP, Intellectual Property Department, Four Embarcadero Center, Suite 3400, San Francisco, CA, 94111-4187, US

(73) Assignee: Xencor, Inc.

(21) Appl. No.: 10/888,348

(22) Filed: Jul. 9, 2004

Related U.S. Application Data
(60) Provisional application No. 60/485,941, filed on Jul. 9, 2003.
(60) Provisional application No. 60/528,229, filed on Dec. 8, 2003.

(51) Int. Cl. C07H 21/04; C12P 21/02; C07K 14/52; C07K 14/475

(52) U.S. Cl. 435/69.5; 435/320.1; 435/325; 530/351; 530/399; 536/23.5

(57) ABSTRACT

The present invention relates to variant ciliary neurotrophic factor (CNTF) proteins that possess neuroprotective and/or weight loss activity and reduced immunogenicty. In particular, variants of CNTF with reduced ability to bind one or more human class 11 MHC molecules are described.

This application claims benefit under 35 U.S.C. §119(e) to U.S. Ser. No. 60/485,941, filed Jul. 9, 2003 and U.S. Ser. No. 60/528,229, filed Dec. 8, 2003, both of which are expressly incorporated by reference in its entirety.

### FIELD OF THE INVENTION

The present invention relates to variant ciliary neurotrophic factor (CNTF) proteins that possess neuroprotective and/or weight loss activity and are substantially non-immunogenic. In particular, variants of CNTF with reduced ability to bind one or more human class II MHC molecules are described.

### BACKGROUND OF THE INVENTION

Immunogenicity is a major barrier to the development and utilization of protein therapeutics. Although immune responses are typically most severe for non-human proteins, even therapeutics based on human proteins can cause immune responses. Immunogenicity is a complex series of responses to a substance that is perceived as foreign and can include production of neutralizing and non-neutralizing antibodies, formation of immune complexes, complement activation, mast cell activation, inflammation, and anaphylaxis.

Several factors can contribute to protein immunogenicity, including but not limited to the protein sequence, the route and frequency of administration, and the patient population.

Immunogenicity may limit the efficacy and safety of a protein therapeutic in multiple ways. Efficacy can be reduced directly by the formation of neutralizing antibodies. Efficacy can also be reduced indirectly, as binding to either neutralizing or non-neutralizing antibodies typically leads to rapid clearance from serum. Severe side effects and even death can occur when an immune reaction is raised. One special class of side effects results when neutralizing antibodies cross-react with an endogenous protein and block its function.

Ciliary neurotrophic factor (CNTF), which was first identified as a neuroprotective cytokine, has attracted significant attention for its ability to promote weight loss. CNTF may act by leptin-like or non-leptin pathways.

However, recent clinical trials of CNTF demonstrated that a large fraction of patients raise neutralizing antibodies against CNTF. These neutralizing antibodies likely decrease the efficacy of the drug. More seriously, neutralizing antibodies could potentially interfere with the neuroprotective functions of endogenous CNTF. Several methods have been developed to modulate the immunogenicity of proteins. In some cases, PEGylation has been observed to reduce the fraction of patients who raise neutralizing antibodies by sterically blocking access to antibody epitopes (see for example, Hershfield et. al. PNAS 1991 88:7185-7189 (1991); Bailon. et al. Bioconjug. Chem. 12: 195-202(2001); He et al. Life Sci. 65: 355-368 (1999)). Methods that improve the solution properties of a protein therapeutic may also reduce immunogenicity, as aggregates have been observed to be more immunogenic than soluble proteins.

A more general approach to immunogenicity reduction involves mutagenesis targeted at the epitopes in the protein sequence and structure that are most responsible for stimulating the immune system. Some success has been achieved by randomly replacing surface-exposed residues to lower binding affinity to panels of known neutralizing antibodies (see for example U.S. Pat. No. 5,766,898). However, due to the incredible diversity of the antibody repertoire, mutations that lower affinity to known antibodies will most likely lead to production of an another set of antibodies rather than abrogation of immunogenicity.

An alternate approach is to disrupt T-cell activation. Removal of MHC-binding epitopes offers a much more tractable approach to immunogenicity reduction, as the diversity of MHC molecules comprises only ˜10³ alleles, while the antibody repertoire is estimated to be approximately 10⁸ and the T-cell receptor repertoire is larger still. By identifying and removing or modifying class II MHC-binding peptides within a protein sequence, the molecular basis of immunogenicity can be evaded. The elimination of such epitopes for the purpose of generating less immunogenic proteins has been disclosed previously; see for example WO 98/52976, WO 02/079232, and WO 00/3317.

Therefore, it is an object of the present invention to provide methods to identify mutations in MHC-binding epitopes confer reduced immunogenicity without resulting in amino acid substitutions that are energetically unfavorable. As a result, the vast majority of the reduced immunogenicity sequences identified using the methods described above are incompatible with the structure and/or function of the protein. In order for MHC epitope removal to be a viable approach for reducing immunogenicity, it is crucial that simultaneous efforts are made to maintain a protein's structure, stability, and biological activity. Accordingly, there is a need to identify less immunogenic variants of CNTF that significantly retain its desired neuroprotective and/or weight loss activity.

### SUMMARY OF THE INVENTION

The present invention relates to variants of CNTF that substantially retain weight loss and/or neuroprotective activity and reduce or substantially eliminate immunogenicity relative to native or commercially relevant CNTF.

An aspect of the present invention are CNTF variants that show decreased binding affinity for one or more class II MHC alleles relative to native or commercially relevant human CNTF and which significantly maintain the weight loss and/or neuroprotective activity of these human CNTFs.

In a further aspect, the invention provides recombinant nucleic acids encoding the variant CNTF proteins, expression vectors, and host cells.

In an additional aspect, the invention provides methods of producing a variant CNTF protein comprising culturing the host cells of the invention under conditions suitable for expression of the variant CNTF protein.

In a further aspect, the invention provides pharmaceutical compositions comprising a variant CNTF protein of the invention and a pharmaceutical carrier.

In a further aspect, the invention provides methods for preventing or treating disorders related to insufficient platelet counts comprising administering a variant CNTF protein of the invention to a patient.

In accordance with the objects outlined above, the present invention provides CNTF variant proteins comprising amino acid sequences with at least one amino acid change compared to the wild type CNTF proteins.

### BRIEF DESCRIPTION OF THE FIGURES

FIG. 1 shows the amino acid sequence of human CNTF. All references to position numbering in the present application are to the naturally occurring human CNTF.

FIG. 2 shows a variant of human CNTF (Axokine®, Regeneron) that has been used in clinical trials. It contains two substitutions as compared to the human wild type and a C-terminal deletion.

FIG. 3 shows a graphical representation of Axokine® (Regeneron) predicted MHC-II binding epitopes.

FIG. 4 shows a graphical representation of Axokine® (Regeneron) predicted epitope 1 tested with an in vitro T-cell proliferation assay (in vitro vaccination—IVV). CD4+ T cells are co-cultured with dendritic cells (DC) pulsed with antigens. EB-5, Epstein-Barr virus peptide (TVFYNIPPMPL); TTD-2, tetanus toxin peptide (FNNFTVSFWLRVPKVSASHLET); Ma, DC and TC from matched donors; Un, DC and TC from unmatched donors; TC and DC represent negative controls in which either cells are cultured exclusively.

FIG. 5 shows uptake of CNTF and immunogenicity estimation using a donor of the following HLA II type: DRB1 *0407 *0701; DQA1 *0201 *030101; DPA1 *0103 *0201; DPB1 *0402 *1701; DQB1 *0202 *0302; DRB4 *0103. Cells were collected and washed prior to fluorescence determination. (5A) Graphical representation of Alexa fluor 488®-labeled protein uptake by dendritic cells. Receptor-mediated endocytosis may be the major mechanism of CNTF uptake (5B) Uptake of Alexa fluor 488®-labeled CNTF followed by fluorescence microscopy. Cells were incubated at 37° C. (5c) Graphical representation of CNTF immunogenicity estimated with the IVV assay. Unmatched, DCs and T cells from different donors; Matched, DCs and T cells from the same donor.

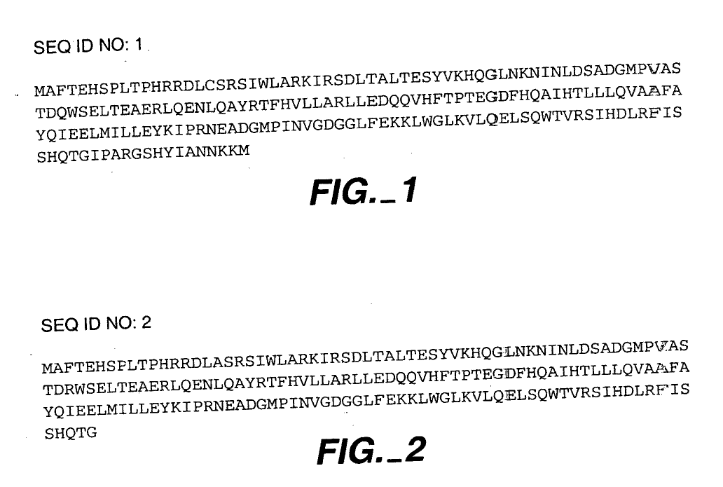

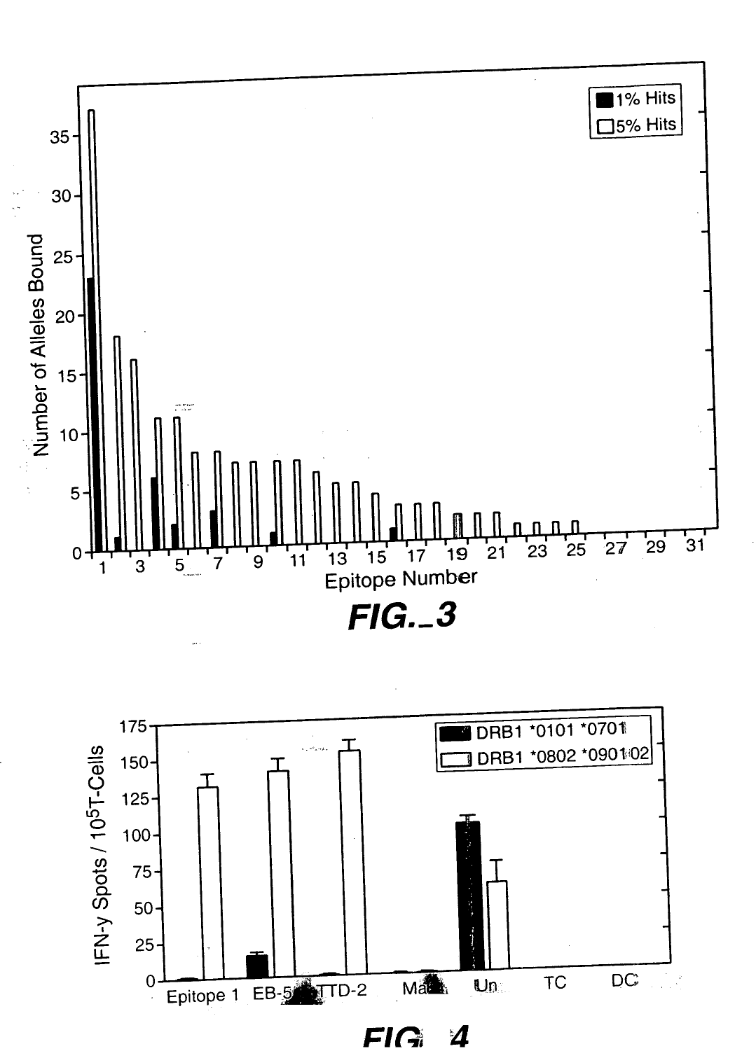

### DETAILED DESCRIPTION OF THE INVENTION

The present invention is directed to methods and compositions that exhibit substantially the same or identical biological activity of naturally occurring or commercially useful human CNTFs but that have reduced binding to class II MHC alleles without a significant loss in stability. In general, the methods involve the identification of putative or actual class II MHC binding domains (e.g. 9-mer frames), preferably that bind to a plurality of class II MHC alleles, which are then altered by amino substitution in such as way as to reduce or destroy binding to the allele while maintaining stability and avoiding increased immunogenicity to the variant.

By “9-mer peptide frame” and grammatical equivalents herein is meant a linear sequence of nine amino acids that is located in a protein of interest. 9-mer frames may be analyzed for their propensity to bind one or more class II MHC alleles.

By “allele” and grammatical equivalents herein is meant an alternative form of a gene. Specifically, in the context of class II MHC molecules, alleles comprise all naturally occurring sequence variants of DRA, DRB1, DRB3/4/5, DQA1, DQB1, DPA1, and DPB1 molecules.

By “hit” and grammatical equivalents herein is meant, in the context of the methods outlined herein (particularly the matrix method), that a given peptide (or 9-mer sequence within the larger CNTF sequence) is predicted to bind to a given class II MHC allele. In a preferred embodiment, a hit is defined to be a peptide sequence with binding affinity among the top 5%, or 3%, or 1% of binding scores of random peptide sequences. In an alternate embodiment, a hit is defined to be a peptide sequence with a binding affinity that exceeds some threshold, for instance a peptide that is predicted to bind an MHC allele with at least 100 uM or 10 uM or 1 uM affinity.

By “immunogenicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis.

By “reduced immunogenicity” and grammatical equivalents herein is meant a decreased ability to activate the immune system, when compared to the wild type protein. For example, a CNTF variant protein can be said to have “reduced immunogenicity” if it elicits neutralizing or non-neutralizing antibodies in lower titer or in fewer patients than wild type CNTF. In a preferred embodiment, the probability of raising neutralizing antibodies is decreased by at least 5%, with at least 50% or 90% decreases being especially preferred. So, if a wild type produces an immune response in 10% of patients, a variant with reduced immunogenicity would produce an immune response in not more than 9.5% of patients, with less than 5% or less than 1% being especially preferred. A CNTF variant protein also can be said to have “reduced immunogenicity” if it shows decreased binding to one or more MHC alleles or if it induces T-cell activation in a decreased fraction of patients relative to wild type CNTF. In a preferred embodiment, the probability of T-cell activation is decreased by at least 5%, with at least 50% or 90% decreases being especially preferred. Particularly preferred in the present invention is the reduction of immungenicity through reduced MHC binding.

By “matrix method” and grammatical equivalents thereof herein is meant a method for calculating peptide sequence—MHC affinity in which a matrix is used that contains a score for each possible residue at each position in the peptide sequence that interacts with a given MHC allele. The binding score for a given peptide is obtained by summing the matrix values for the amino acids observed at each position in the peptide. In a preferred embodiment, the matrix values (e.g. scores) are calculated using a computational method outlined herein.

By “MHC-binding epitopes” and grammatical equivalents herein is meant peptides that are capable of binding to one or more class II MHC alleles with appropriate affinity to enable the formation of a MHC—peptide—T-cell receptor complex and subsequent T-cell activation. MHC-binding epitopes are linear peptides that comprise at least approximately 9 residues.

By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992)], generally depending on the method of synthesis. For example, homo-phenylalanine, citrulline, and noreleucine are considered amino acids for the purposes of the invention. “Amino acid” also includes amino acid residues such as proline and hydroxyproline. Both D- and L-amino acids may be utilized.

By “CNTF-responsive disorders or conditions” and grammatical equivalents herein is meant diseases, disorders, and conditions that can benefit from treatment with CNTF. Examples of CNTF-responsive disorders include, but are not limited to, obesity, diabetes, pre-diabetes, stroke, motor neuron diseases including amylotrophic lateral sclerosis.

By “treatment” herein is meant to include therapeutic treatment, as well as prophylactic, or suppressive measures for the disease or disorder. Thus, for example, successful administration of a variant CNTF protein prior to onset of the disease may result in treatment of the disease. As another example, successful administration of a variant CNTF protein after clinical manifestation of the disease to combat the symptoms of the disease comprises “treatment” of the disease. “Treatment” also encompasses administration of a variant CNTF protein after the appearance of the disease in order to eradicate the disease. Successful administration of an agent after onset and after clinical symptoms have developed, with possible abatement of clinical symptoms and perhaps amelioration of the disease, further comprises “treatment” of the disease. Those “in need of treatment” include mammals already having the disease or disorder, as well as those prone to having the disease or disorder, including those in which the disease or disorder is to be prevented.

By “therapeutically effective dose” herein is meant a dose that produces the effects for which it is administered. The exact dose will depend on the purpose of the treatment, and will be ascertainable by one skilled in the art using known techniques. In a preferred embodiment, dosages of about 5 μg/kg are used, administered either intravenously or subcutaneously. As is known in the art, adjustments for variant CNTF protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

By “patient” herein is meant both humans and other animals, particularly mammals, and organisms. Thus the methods are applicable to both human therapy and veterinary applications. In the preferred embodiment the patient is a mammal, and in the most preferred embodiment the patient is human.

By “variant CNTF proteins” and grammatical equivalents thereof herein is meant non-naturally occurring CNTF proteins which differ from the wild type CNTF protein by at least 1 amino acid insertion, deletion, or substitution, and generally have reduced immunogenicity as compared to a wild-type CNTF. CNTF variants are characterized by the predetermined nature of the variation, a feature that sets them apart from naturally occurring allelic or interspecies variation of the CNTF protein sequence. The CNTF variants typically either exhibit the same qualitative biological activity as the naturally occurring CNTF or have been specifically engineered to have alternate biological properties. The variant CNTF proteins may contain insertions, deletions, and/or substitutions at the N-terminus, C-terminus, or internally. In a preferred embodiment, variant CNTF proteins have at least 1 residue that differs from the human CNTF sequence, with at least 2, 3, 4, or 5 different residues being more preferred. In an alternate preferred embodiment, the CNTF variants comprise a 13 or 15 residue C-terminal deletion. In another preferred embodiment, the CNTF variants comprise the substitutions C17A and/or Q63R and/or the C-terminal deletion. Variant CNTF proteins may contain further modifications, for instance mutations that alter stability or solubility or which enable or prevent posttranslational modifications such as PEGylation or glycosylation. Variant CNTF proteins may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels.

By “wild-type” and grammatical equivalents thereof herein is meant an amino acid sequence or a nucleotide sequence that is found in nature and includes allelic variations; that is, an amino acid sequence or a nucleotide sequence that has not been intentionally modified. In a preferred embodiment, the wild-type sequence is the most prevalent human sequence. However, the wild type CNTF proteins may be from any number of organisms, include, but are not limited to, rodents (rats, mice, hamsters, guinea pigs, etc.), primates, and farm animals (including sheep, goats, pigs, cows, horses, etc). As will be appreciated by those in the art, CNTF proteins from mammals other than humans may find use in animal models of human disease.

“Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Identification of MHC-Binding Epitopes in CNTF

In general, the present invention is directed to the alteration of amino acids within MHC binding epitopes in CNTF proteins that decrease the binding of the epitope peptide to the class II MHC alleles, yet retain biological activity and stability, such that upon administration of the variant, biological activity and stability is seen with reduced or eliminated immunogenicity and it's corresponding problems. MHC-binding peptides are obtained from proteins such as CNTF by a process called antigen processing. First, the protein is transported into an antigen presenting cell by endocytosis or phagocytosis. A variety of proteolytic enzymes then cleave the protein into a number of peptides. Next, these peptides can then be loaded onto class II MHC molecules, and the resulting peptide-MHC complexes are transported to the cell surface. Relatively stable peptide-MHC complexes can be recognized by T-cell receptors that are present on the surface of naïve T-cells. This recognition event is required for the initiation of an immune response. Accordingly, blocking the formation of stable peptide-MHC complexes is an effective approach for preventing unwanted immune responses.

The factors that determine the affinity of peptide-MHC interactions have been characterized using biochemical and structural methods. Peptides bind in an extended conformation bind along a groove in the class II MHC molecule. While peptides that bind class II MHC molecules are typically approximately 13-18 residues long, a nine-residue region is responsible for most of the binding affinity and specificity. The peptide binding groove can be subdivided into “pockets”, commonly named P1 through P9, where each pocket is comprises the set of MHC residues that interacts with a specific residue in the peptide. A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding epitopes in protein sequences are known in the art and may be used to identify epitopes in CNTF.

Sequence-based information can be used to determine a binding score for a given peptide—MHC interaction (see for example Mallios, Bioinformatics 15: 432-439 (1999); Mallios, Bioinformatics 17: p942-948 (2001); Sturniolo et. al. Nature Biotech. 17: 555-561(1999)). It is possible to use structure-based methods in which a given peptide is computationally placed in the peptide-binding groove of a given MHC molecule and the interaction energy is determined (for example, see WO 98/59244 and WO 02/069232). Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (see for example WO 02/77187).

In a preferred embodiment, MHC-binding propensity scores are calculated for each 9-residue frame along the human CNTF sequence using a matrix method (see Sturniolo et. al., supra; Marshall et. al., J. Immunol. 154: 5927-5933 (1995), and Hammer et. al., J. Exp. Med. 180: 2353-2358 (1994), all of which are expressly incorporated by reference in their entirety). The matrix comprises binding scores for specific amino acids interacting with the peptide binding pockets in different human class II MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket. Matrices that are produced by extrapolation are referred to as “virtual matrices”.

In a preferred embodiment, the matrix method is used to calculate scores for each peptide of interest binding to each allele of interest. Several methods can then be used to determine whether a given peptide will bind with significant affinity to a given MHC allele. In one embodiment, the binding score for the peptide of interest is compared with the binding propensity scores of a large set of reference peptides. Peptides whose binding propensity scores are large compared to the reference peptides are likely to bind MHC and may be classified as “hits”. For example, if the binding propensity score is among the highest 1% of possible binding scores for that allele, it may be scored as a “hit” at the 1% threshold. The total number of hits at one or more threshold values is calculated for each peptide. In some cases, the binding score may directly correspond with a predicted binding affinity. Then, a hit may be defined as a peptide predicted to bind with at least 100 □M or 10 □M or 1 □M affinity.

In a preferred embodiment, the number of hits for each 9-mer frame in the protein is calculated using one or more threshold values ranging from 0.5% to 10%. In an especially preferred embodiment, the number of hits is calculated using 1%, 3%, and 5% thresholds.

In a preferred embodiment, MHC-binding epitopes are identified as the 9-mer frames that bind to several class II MHC alleles. In an especially preferred embodiment, MHC-binding epitopes are predicted to bind at least 10 alleles at 5% threshold and/or at least 5 alleles at 1% threshold. Such 9-mer frames may be especially likely to elicit an immune response in many members of the human population.

In an alternate preferred embodiment, MHC-binding epitopes are predicted to bind MHC alleles that are present in at least 0.01-10% of the human population.

In an additional preferred embodiment, MHC-binding epitopes are identified as the 9-mer frames that are located among “nested” epitopes, or overlapping 9-residue frames that are each predicted to bind a significant number of alleles. Such sequences may be especially likely to elicit an immune response.

Preferred MHC-binding epitopes in CNTF include, but are not limited to, residues 21-29, 27-35, 77-85, 80-88, and 176-184.

Especially preferred MHC-binding epitopes in native CNTF include, but are not limited to, residues 80-88. This epitopes is predicted to bind to a large number of MHC alleles. Furthermore, this epitope is located within a region of nested epitopes; for example, residues 80-88 overlap with the immunogenic epitopes at residues77-85 and 83-91.

Confirmation of MHC-Binding Epitopes

In a preferred embodiment, the immunogenicity of the above-predicted MHC-binding epitopes is experimentally confirmed by measuring the extent to which peptides comprising each predicted epitope can elicit an immune response. However, it is possible to proceed from epitope prediction to epitope removal without the intermediate step of epitope confirmation. Epitope removal is general accomplished by substituting at least one amino acid within the epitope, e.g. the 9-mer, as outlined below

Several methods, discussed in more detail below, can be used for experimental confirmation of epitopes. For example, sets of naïve T-cells and antigen presenting cells from matched donors can be stimulated with a peptide containing an epitope of interest, and T-cell activation can be monitored. In a preferred embodiment, interferon gamma production by activated T-cells is monitored, although it is also possible to use other indicators of T-cell activation or proliferation such as tritiated thymidine incorporation or interleukin 5 (IL5) production.

Design of Active, Less-Immunogenic Variants

In a preferred embodiment, the above-determined MHC-binding epitopes are replaced with alternate amino acid sequences to generate active variant CNTF proteins with reduced or eliminated immunogenicity, particularly through reduced MHC binding. There are several possible strategies for integrating methods for identifying less immunogenic sequences with methods for identifying structured and active sequences, including but not limited to those presented below.

Protein design methods and MHC epitope identification methods can be used together to identify stable, active, and minimally immunogenic protein sequences (see WO 03/006154 hereby incorporated by reference in its entirety). The combination of approaches provides significant advantages over the prior art for immunogenicity reduction, as most of the reduced immunogenicity sequences identified using other techniques fail to retain sufficient activity and stability to serve as therapeutics.

A wide variety of methods are known for generating and evaluating sequences. These include, but are not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994)) all of which are expressly incorporated by reference herein.

In a preferred embodiment, rational design of novel CNTF variants is achieved by using Protein Design Automation® (PDA®) technology. (See U.S. Pat. Nos. 6,188,965; 6,269,312; 6,403,312; WO 98/47089 and U.S. Ser. Nos. 09/058,459, 09/127,926, 60/104,612, 60/158,700, 09/419,351, 60/181,630, 60/186,904, 09/419,351, 09/782,004 and 09/927,790, 60/347,772, and 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all references expressly incorporated herein in their entirety.)

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions. PDA® technology has been applied to numerous systems including important pharmaceutical and industrial proteins and has a demonstrated record of success in protein optimization.

PDA® utilizes three-dimensional structural information. In the most preferred embodiment, the structure of CNTF is obtained by solving its crystal structure or NMR structure by techniques well known in the art. For example, the crystal structure with PDB accession code 1CNT can be used (see McDonald, N. Q., Panayotatos, N. and Hendrickson, W. A., EMBO J. 14: 2689-2699 (1995)).

In a preferred embodiment, the results of matrix method calculations are used to identify which of the 9 amino acid positions within the epitope(s) contribute most to the overall binding propensities for each particular allele “hit”. This analysis considers which positions (P1-P9) are occupied by amino acids which consistently make a significant contribution to MHC binding affinity for the alleles scoring above the threshold values. Matrix method calculations are then used to identify amino acid substitutions at said positions that would decrease or eliminate predicted immunogenicity and PDA® technology is used to determine which of the alternate sequences with reduced or eliminated immunogenicity are compatible with maintaining the structure and function of the protein.

Alternatively, substitutions at each of the 9 amino acid positions can be done, followed by the matrix method calculations to identify good substitutions, with PDA® technology being used to identify the best energetically favorable sequences.

In an alternate preferred embodiment, the residues in each epitope are first analyzed by one skilled in the art to identify alternate residues that are potentially compatible with maintaining the structure and function of the protein. This may be done in a variety of ways. In one embodiment, each residue position is classified as a core residue, a surface residue, or a boundary residue, and each classification defines a subset of possible amino acid residues for the position (for example, core residues generally will be selected from the set of hydrophobic residues, surface residues generally will be selected from the hydrophilic residues, and boundary residues may be either). Then, the set of resulting sequences are computationally screened to identify the least immunogenic variants. Finally, each of the less immunogenic sequences are analyzed more thoroughly in PDA® technology protein design calculations to identify protein sequences that maintain the protein structure and function and decrease immunogenicity.

In an alternate preferred embodiment, each residue that contributes significantly to the MHC binding affinity of an epitope is analyzed to identify a subset of amino acid substitutions that maintain the structure and function of the protein. This step may be performed in several ways, including PDA® calculations or visual inspection by one skilled in the art. Sequences may be generated that contain all possible combinations of amino acids that were selected for consideration at each position, or, in some cases, all possible substitutions can be done. Matrix method calculations can be used to determine the immunogenicity of each sequence. The results can be analyzed to identify sequences that have significantly decreased immunogenicity. Additional PDA® calculations may be performed to determine which of the minimally immunogenic sequences are compatible with maintaining the structure and function of the protein.

In an alternate preferred embodiment, pseudo-energy terms derived from the peptide binding propensity matrices are incorporated directly into the PDA® technology calculations. In this way, it is possible to select sequences that are active and less immunogenic in a single computational step.

In a preferred embodiment, PDA® technology and matrix method calculations are used to remove more than one MHC-binding epitope from a protein of interest.

Generating the Variants

Variant CNTF proteins of the invention and nucleic acids encoding them may be produced using a number of methods known in the art. Generally, this involves synthesizing nucleic acid sequences encoding the desired sequences. In general, for methods utilizing the just the variant 9-mer peptide sequences, the sequences are made synthetically. Alternatively, for longer (e.g. full length CNTF variants comprising the variant sequences), traditional recombinant methods can be used.

In a preferred embodiment, CNTF variants are cloned into an appropriate expression vector and expressed in E. coli (see McDonald, J. R., Ko, C., Mismer, D., Smith, D. J. and Collins, F. Biochim. Biophys. Acta 1090: 70-80 (1991)). Bacterial expression systems and methods for their use are well known in the art (see Current Protocols in Molecular Biology, Wiley & Sons, and Molecular Cloning—A Laboratory Manual—3^{rd} Ed., Cold Spring Harbor Laboratory Press, New York (2001)). The choice of codons, suitable expression vectors and suitable host cells will vary depending on a number of factors, and may be easily optimized as needed.

In an alternate preferred embodiment, CNTF variants are expressed in mammalian cells, yeast, baculovirus, or in vitro expression systems. Any number of known methods can be used, as outlined in the methods of the incorporated references.

In a preferred embodiment, the CNTF variants are purified or isolated after expression. Standard purification methods include electrophoretic, molecular, immunological and chromatographic techniques, including ion exchange, hydrophobic, affinity, and reverse-phase HPLC chromatography, and chromatofocusing. For example, a CNTF variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3r ed (1994). The degree of purification necessary will vary depending on the desired use, and in some instances no purification will be necessary.

Assaying the Activity of the Variants

Variant sequences that are designed to maintain weight loss and/or neuroprotective activity and to have reduced or eliminated immunogenicity may be tested experimentally for activity. As is known to those in the art, several methods may be used to characterize the activity of CNTF and CNTF variants.

In one preferred embodiment, any of a variety of receptor binding assays can be used. CNTF's receptors include, but are not limited to, CNTFR□, gp130, and LIFR. These assays are described in the incorporated references.

In another preferred embodiment, a reporter gene assay is used. For example, activation of STAT transcription factors can be monitored. In general, this involves utilizing a biological activity of CNTF, such as activation of STAT transcription factors, is done, in some cases using cells containing reporter genes hooked to the binding partners of one or more of the transcription factors.

In another preferred embodiment, animal studies are conducted to characterize the weight loss of the CNTF variants (see for example Gloaguen et. al. Proc. Nat. Acad. Sci. USA 94: 6456-6461 (1997), the methodologies of which are incorporated by references).

In another preferred embodiment, neuroprotective activity of the CNTF variants is characterized by measuring neuronal survival (see for example Inoue et. al. Proc. Nat. Acad. Sci. USA 92: 8579-8583 (1995) the methodologies of which are incorporated by references).

Determining the Immunogenicity of the Variants

In a preferred embodiment, the immunogenicity of the CNTF variants is determined experimentally to confirm that the variants do have reduced or eliminated immunogenicity relative to the wild type protein.

In a preferred embodiment, ex vivo T cell activation assays are used to experimentally quantitate immunogenicity. In this method, antigen presenting cells and naïve T cells from matched donors are challenged with a variant peptide of the invention (e.g. a 9-mer) or whole CNTF protein containing variant sequence, one or more times. Then, T cell activation can be detected using a number of methods, for example by monitoring production of cytokines or measuring uptake of tritiated thymidine. In the most preferred embodiment, interferon gamma production is monitored using Elispot assays (see Schmittel et. al. J. Immunol. Meth., 24: 17-24 (2000), the methodologies of which are incorporated by reference.

In an alternate preferred embodiment, immunogenicity is measured in transgenic mouse systems. For example, mice expressing fully or partially human class II MHC molecules may be used.

In an alternate embodiment, immunogenicity is tested by administering the CNTF variants to one or more animals, including rodents and primates, and monitoring for antibody formation.

Once made, the variant CNTF proteins and nucleic acids of the invention find use in a number of applications. In a preferred embodiment, the variant CNTF proteins are administered to a patient to treat a CNTF related disorder.

The administration of the variant CNTF proteins of the present invention, preferably in the form of a sterile aqueous solution, may be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, the variant CNTF protein may be directly applied as a solution or spray. Depending upon the manner of introduction, the pharmaceutical composition may be formulated in a variety of ways. The concentration of the therapeutically active variant CNTF protein in the formulation may vary from about 0.1 to 100 weight %. In another preferred embodiment, the concentration of the variant CNTF protein is in the range of 0.003 to 1.0 molar, with dosages from 0.03, 0.05, 0.1, 0.2, and 0.3 millimoles per kilogram of body weight being preferred.

The pharmaceutical compositions of the present invention comprise a variant CNTF protein in a form suitable for administration to a patient. In a preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the variant TNF-alpha proteins are added in a micellular formulation; see U.S. Pat. No. 5,833,948, hereby expressly incorporated by reference in its entirety.

Combinations of pharmaceutical compositions may be administered. Moreover, the compositions may be administered in combination with other therapeutics.

In one embodiment provided herein, antibodies, including but not limited to monoclonal and polyclonal antibodies, are raised against variant CNTF proteins using methods known in the art. In a preferred embodiment, these anti-variant CNTF antibodies are used for immunotherapy. Thus, methods of immunotherapy are provided. By “immunotherapy” is meant treatment of a CNTF related disorders with an antibody raised against a CNTF protein. As used herein, immunotherapy can be passive or active. Passive immunotherapy, as defined herein, is the passive transfer of antibody to a recipient (patient). Active immunization is the induction of antibody and/or T-cell responses in a recipient (patient).

### EXAMPLES

### Example 1

### Identification of MHC-Bindinq Epitopes in CNTF

In order to find MHC-binding epitopes, each 9-residue fragment of native human CNTF was analyzed for its propensity to bind to each of 52 class II MHC alleles for which peptide binding affinity matrices have been derived. The calculations were performed using cutoffs of 1% ,3% and 5%. The number of alleles that each peptide is predicted to bind at each of these cutoffs are shown below. 9-mer peptides that are not listed below are not any alleles at the 5%, 3%, or 1% cutoffs.

| First | Last | | | | |
|---|---|---|---|---|---|
| Residue | Residue | Sequence | 1% Hits | 3% Hits | 5% Hits |
| 16 | 24 | LCSRSIWLA | 0 | 0 | 1 |
| 21 | 29 | IWLARKIRS | 0 | 5 | 16 |
| 22 | 30 | WLARKIRSD | 1 | 2 | 3 |
| 23 | 31 | LARKIRSDL | 0 | 0 | 1 |
| 27 | 35 | IRSDLTALT | 6 | 11 | 11 |
| 38 | 46 | YVKHQGLNK | 0 | 7 | 7 |
| 44 | 52 | LNKNINLDS | 0 | 4 | 6 |
| 48 | 56 | INLDSADGM | 0 | 6 | 8 |
| 77 | 85 | LQAYRTFHV | 2 | 3 | 11 |
| 80 | 88 | YRTFHVLLA | 23 | 34 | 37 |
| 83 | 91 | FHVLLARLL | 3 | 4 | 8 |
| 85 | 93 | VLLARLLED | 0 | 2 | 3 |
| 112 | 120 | LLLQVAAFA | 0 | 1 | 5 |
| 113 | 121 | LLQVAAFAY | 0 | 2 | 2 |
| 121 | 129 | YQIEELMIL | 0 | 6 | 7 |
| 126 | 134 | LMILLEYKI | 0 | 2 | 2 |
| 130 | 138 | LEYKIPRNE | 1 | 3 | 7 |
| 132 | 140 | YKIPRNEAD | 0 | 0 | 1 |
| 156 | 164 | LWGLKVLQE | 0 | 2 | 4 |
| 157 | 165 | WGLKVLQEL | 0 | 0 | 3 |
| 159 | 167 | LKVLQELSQ | 0 | 3 | 5 |
| 165 | 173 | LSQWTVRSI | 0 | 1 | 7 |
| 168 | 176 | WTVRSIHDL | 0 | 0 | 1 |
| 170 | 178 | VRSIHDLRF | 0 | 0 | 2 |
| 176 | 184 | LRFISSHQT | 1 | 12 | 18 |
| 178 | 186 | FISSHQTGI | 0 | 2 | 2 |

Based on the above analysis, the 9-mer residues that are predicted to bind to the most MHC alleles are residues 21-29, 27-35, 77-85, 80-88, and 176-184.

The analysis was repeated for the CNTF variant Axokine®; the location of the epitopes is the same for the two proteins.

### Example 2

### Identification of Less Immunogenic Variants

In preferrd embodiment, each position that contributes to MHC binding is analyzed to identify a sudset of amino acid substitutions that are potentially compatible with maintaining the structure and function of the protein. This step may be performed in several ways, including PDA® calculations or visual inspection by one skilled in the art. Sequences may be generated that contain all possible combinations of amino acids that were selected for consideration at each position. Matrix method calculations can be used to determine the immunogenicity each sequence. The results can be analyzed to identify sequences that have significantly decreased immunogenicity. Additional PDA® calculations may be performed to determine which of the minimally immunogenic sequences are compatible with maintaining the structure and function of the protein.

| sequence | anchor1% | anchor3% | anchor5% | overlap1% | overlap3% | overlap5% |
|---|---|---|---|---|---|---|
| YRTFHVLLA | 23 | 34 | 37 | 5 | 9 | 22 |
| YEEFHQRLA | 0 | 0 | 0 | 0 | 0 | 0 |
| YKEFHQRLA | 0 | 0 | 0 | 0 | 0 | 0 |
| YQEFHQRLA | 0 | 0 | 0 | 0 | 0 | 0 |
| LEEFHARLA | 0 | 0 | 0 | 0 | 0 | 0 |
| LEEFHQRLA | 0 | 0 | 0 | 0 | 0 | 0 |
| LEELHAELA | 0 | 0 | 0 | 0 | 0 | 0 |
| LEELHAKLA | 0 | 0 | 0 | 0 | 0 | 0 |
| LEQFHARLA | 0 | 0 | 0 | 0 | 0 | 0 |
| LKEFHARLA | 0 | 0 | 0 | 0 | 0 | 0 |
| LKEFHQRLA | 0 | 0 | 0 | 0 | 0 | 0 |
| LKELHAELA | 0 | 0 | 0 | 0 | 0 | 0 |
| LKELHAKLA | 0 | 0 | 0 | 0 | 0 | 0 |
| LQEFHARLA | 0 | 0 | 0 | 0 | 0 | 0 |
| LQEFHQRLA | 0 | 0 | 0 | 0 | 0 | 0 |
| LQELHAELA | 0 | 0 | 0 | 0 | 0 | 0 |
| LQELHAKLA | 0 | 0 | 0 | 0 | 0 | 0 |
| YREFHQELA | 0 | 0 | 0 | 0 | 0 | 1 |
| YREFHQQLA | 0 | 0 | 0 | 0 | 1 | 1 |
| YRELHQELA | 0 | 0 | 0 | 0 | 0 | 1 |
| YRELHQKLA | 0 | 0 | 0 | 0 | 0 | 1 |
| YEEFHQELA | 0 | 0 | 0 | 0 | 0 | 1 |
| YEEFHQQLA | 0 | 0 | 0 | 0 | 1 | 1 |
| YEELHQELA | 0 | 0 | 0 | 0 | 0 | 1 |
| YEELHQKLA | 0 | 0 | 0 | 0 | 0 | 1 |
| YKEFHQELA | 0 | 0 | 0 | 0 | 0 | 1 |
| YKEFHQQLA | 0 | 0 | 0 | 0 | 1 | 1 |
| YKELHQELA | 0 | 0 | 0 | 0 | 0 | 1 |
| YKELHQKLA | 0 | 0 | 0 | 0 | 0 | 1 |
| YQEFHQELA | 0 | 0 | 0 | 0 | 0 | 1 |
| YQEFHQQLA | 0 | 0 | 0 | 0 | 1 | 1 |
| YQELHQELA | 0 | 0 | 0 | 0 | 0 | 1 |
| YQELHQKLA | 0 | 0 | 0 | 0 | 0 | 1 |
| LREFHAELA | 0 | 0 | 0 | 0 | 0 | 1 |
| LREFHQELA | 0 | 0 | 0 | 0 | 0 | 1 |
| LREFHQQLA | 0 | 0 | 0 | 0 | 1 | 1 |
| LEEFHAELA | 0 | 0 | 0 | 0 | 0 | 1 |
| LEEFHAQLA | 0 | 0 | 0 | 0 | 1 | 1 |
| LEEFHQELA | 0 | 0 | 0 | 0 | 0 | 1 |
| LEEFHQQLA | 0 | 0 | 0 | 0 | 1 | 1 |
| LEELHAQLA | 0 | 0 | 0 | 0 | 0 | 1 |
| LEELHARLA | 0 | 0 | 0 | 0 | 0 | 1 |
| LEQFHAELA | 0 | 0 | 0 | 0 | 0 | 1 |
| LEQFHAQLA | 0 | 0 | 0 | 0 | 1 | 1 |
| LKEFHAELA | 0 | 0 | 0 | 0 | 0 | 1 |
| LKEFHAQLA | 0 | 0 | 0 | 0 | 1 | 1 |
| LKEFHQELA | 0 | 0 | 0 | 0 | 0 | 1 |
| LKEFHQQLA | 0 | 0 | 0 | 0 | 1 | 1 |
| LKELHAQLA | 0 | 0 | 0 | 0 | 0 | 1 |
| LKELHARLA | 0 | 0 | 0 | 0 | 0 | 1 |
| LKQFHAELA | 0 | 0 | 0 | 0 | 0 | 1 |
| LQEFHAELA | 0 | 0 | 0 | 0 | 0 | 1 |
| LQEFHAQLA | 0 | 0 | 0 | 0 | 1 | 1 |
| LQEFHQELA | 0 | 0 | 0 | 0 | 0 | 1 |
| LQEFHQQLA | 0 | 0 | 0 | 0 | 1 | 1 |
| LQELHAQLA | 0 | 0 | 0 | 0 | 0 | 1 |
| LQELHARLA | 0 | 0 | 0 | 0 | 0 | 1 |
| LQQFHAELA | 0 | 0 | 0 | 0 | 0 | 1 |
| YREFHQKLA | 0 | 0 | 0 | 0 | 0 | 2 |
| YRELHQQLA | 0 | 0 | 0 | 0 | 0 | 2 |
| YEEFHARLA | 0 | 0 | 0 | 0 | 0 | 2 |
| YEEFHQKLA | 0 | 0 | 0 | 0 | 0 | 2 |
| YEELHQQLA | 0 | 0 | 0 | 0 | 0 | 2 |
| YEELHQRLA | 0 | 0 | 0 | 0 | 0 | 2 |
| YKEFHQKLA | 0 | 0 | 0 | 0 | 0 | 2 |
| YKELHQQLA | 0 | 0 | 0 | 0 | 0 | 2 |
| YKELHQRLA | 0 | 0 | 0 | 0 | 0 | 2 |
| YQEFHQKLA | 0 | 0 | 0 | 0 | 0 | 2 |
| YQELHQQLA | 0 | 0 | 0 | 0 | 0 | 2 |
| YQELHQRLA | 0 | 0 | 0 | 0 | 0 | 2 |
| LREFHVELA | 0 | 0 | 0 | 0 | 1 | 2 |
| LREFHAKLA | 0 | 0 | 0 | 0 | 0 | 2 |
| LREFHQKLA | 0 | 0 | 0 | 0 | 0 | 2 |
| LRELHVELA | 0 | 0 | 0 | 0 | 0 | 2 |
| LEAFHARLA | 0 | 0 | 0 | 0 | 2 | 2 |
| LEEFHVELA | 0 | 0 | 0 | 0 | 1 | 2 |
| LEEFHAKLA | 0 | 0 | 0 | 0 | 0 | 2 |
| LEEFHQKLA | 0 | 0 | 0 | 0 | 0 | 2 |
| LEELHVELA | 0 | 0 | 0 | 0 | 0 | 2 |
| LEQFHVELA | 0 | 0 | 0 | 0 | 1 | 2 |
| LEQFHAKLA | 0 | 0 | 0 | 0 | 0 | 2 |
| LKEFHVELA | 0 | 0 | 0 | 0 | 1 | 2 |
| LKEFHAKLA | 0 | 0 | 0 | 0 | 0 | 2 |
| LKEFHQKLA | 0 | 0 | 0 | 0 | 0 | 2 |
| LKELHVELA | 0 | 0 | 0 | 0 | 0 | 2 |
| LKQFHAKLA | 0 | 0 | 0 | 0 | 0 | 2 |
| LQEFHVELA | 0 | 0 | 0 | 0 | 1 | 2 |
| LQEFHAKLA | 0 | 0 | 0 | 0 | 0 | 2 |
| LQEFHQKLA | 0 | 0 | 0 | 0 | 0 | 2 |
| LQELHVELA | 0 | 0 | 0 | 0 | 0 | 2 |
| LQQFHAKLA | 0 | 0 | 0 | 0 | 0 | 2 |
| YREFHAELA | 0 | 0 | 0 | 0 | 0 | 3 |
| YEEFHAELA | 0 | 0 | 0 | 0 | 0 | 3 |
| YEEFHAQLA | 0 | 0 | 0 | 0 | 1 | 3 |
| YEELHAELA | 0 | 0 | 0 | 0 | 2 | 3 |
| YEELHAKLA | 0 | 0 | 0 | 0 | 2 | 3 |
| YKEFHAELA | 0 | 0 | 0 | 0 | 0 | 3 |
| YKEFHAQLA | 0 | 0 | 0 | 0 | 1 | 3 |
| YKELHAELA | 0 | 0 | 0 | 0 | 2 | 3 |
| YKELHAKLA | 0 | 0 | 0 | 0 | 2 | 3 |
| YQEFHAELA | 0 | 0 | 0 | 0 | 0 | 3 |
| YQEFHAQLA | 0 | 0 | 0 | 0 | 1 | 3 |
| YQELHAELA | 0 | 0 | 0 | 0 | 2 | 3 |
| YQELHAKLA | 0 | 0 | 0 | 0 | 2 | 3 |
| LRELHLELA | 0 | 0 | 0 | 0 | 1 | 3 |
| LRELHQELA | 0 | 0 | 0 | 0 | 0 | 3 |
| LRELHQKLA | 0 | 0 | 0 | 0 | 0 | 3 |
| LEAFHAELA | 0 | 0 | 0 | 0 | 2 | 3 |
| LEAFHAQLA | 0 | 0 | 0 | 0 | 3 | 3 |
| LEELHLELA | 0 | 0 | 0 | 0 | 1 | 3 |
| LEELHQELA | 0 | 0 | 0 | 0 | 0 | 3 |
| LEELHQKLA | 0 | 0 | 0 | 0 | 0 | 3 |
| LKAFHAELA | 0 | 0 | 0 | 0 | 2 | 3 |
| LKELHLELA | 0 | 0 | 0 | 0 | 1 | 3 |
| LKELHQELA | 0 | 0 | 0 | 0 | 0 | 3 |
| LKELHQKLA | 0 | 0 | 0 | 0 | 0 | 3 |
| LQAFHAELA | 0 | 0 | 0 | 0 | 2 | 3 |
| LQELHLELA | 0 | 0 | 0 | 0 | 1 | 3 |
| LQELHQELA | 0 | 0 | 0 | 0 | 0 | 3 |
| LQELHQKLA | 0 | 0 | 0 | 0 | 0 | 3 |
| LRELHAELA | 0 | 0 | 1 | 0 | 0 | 0 |
| LRELHAKLA | 0 | 0 | 1 | 0 | 0 | 0 |
| LREFHAQLA | 0 | 0 | 1 | 0 | 1 | 1 |
| LKQFHAQLA | 0 | 0 | 2 | 0 | 1 | 1 |
| LQQFHAQLA | 0 | 0 | 2 | 0 | 1 | 1 |
| YKEFHARLA | 0 | 0 | 2 | 0 | 0 | 2 |
| YQEFHARLA | 0 | 0 | 2 | 0 | 0 | 2 |
| LKQFHVELA | 0 | 0 | 2 | 0 | 1 | 2 |
| LQQFHVELA | 0 | 0 | 2 | 0 | 1 | 2 |
| YEQFHARLA | 0 | 0 | 2 | 0 | 2 | 3 |
| LKAFHAQLA | 0 | 0 | 2 | 0 | 3 | 3 |
| LQAFHAQLA | 0 | 0 | 2 | 0 | 3 | 3 |
| LREFHQRLA | 0 | 0 | 3 | 0 | 0 | 0 |
| YRELHAELA | 0 | 1 | 1 | 0 | 2 | 3 |
| LRELHAQLA | 0 | 1 | 2 | 0 | 0 | 1 |
| YREFHAQLA | 0 | 1 | 2 | 0 | 1 | 3 |
| YRELHAKLA | 0 | 1 | 2 | 0 | 2 | 3 |
| YRELHQRLA | 0 | 2 | 3 | 0 | 0 | 2 |

Using the above-preferred embodiment, sequences were identified for the residue 80-88 epitope. These sequences eliminate all or most of the hits in the 80-88 epitope and also eliminate all or nearly all of the hits in the overlapping epitopes. The wild-type sequence and scores are shown in the top row of data for reference. In all of the variants shown below, it is possible to replace Y80 with alternate non-hydrophobic residues, including D, E, G, H, K, N, Q, R, S, and T.

### Example 3

### Identification of Structured, Less Immunogenic CNTF Variants

PDA® calculations were performed to predict the energies of each of the less immunogenic variants of the major epitopes in CNTF, as well as the native sequence. The energies of the native sequences were then compared with the energies of the variants to determine which of the less immunogenic CNTF sequences are compatible with maintaining the structure and function of CNTF. Unless otherwise noted, the nine residues comprising an epitope of interest were determined to be the variable residue positions. Coordinates for the CNTF template were obtained from PDB accession code 1CNT. A variety of rotameric states were considered for each variable position, and the sequence was constrained to be the sequence of a specific less immunogenic variant identified previously. Rotamer-template and rotamer-rotamer energies were then calculated using a force field including terms describing van der Waals interactions, hydrogen bonds, electrostatics, and solvation. The optimal rotameric configurations for each sequence were determined using DEE as a combinatorial optimization method.

In general, all of the sequences whose energies are similar to or better than (that is, less than) the energy of the native sequence are likely to be structured. Sequences that conserve those residues that are known to be important for function are likely to also be active. Alternatively, it is possible to experimentally determine or model the interaction of CNTF with its receptors and then to determine which variant sequences are compatible with forming this interaction.

Less immunogenic CNTF variants that are predicted to be compatible with maintaining the structure and function of CNTF include, but are not limited to, the following:

| sequence | energy | anchor1% | anchor3% | anchor5% | overlap1% | overlap3% | overlap5% |
|---|---|---|---|---|---|---|---|
| YRTFHVLLA | −63.60 | 23 | 34 | 37 | 5 | 9 | 22 |
| YEEFHARLA | −77.63 | 0 | 0 | 0 | 0 | 0 | 2 |
| YEQFHARLA | −75.51 | 0 | 0 | 2 | 0 | 2 | 3 |
| YEEFHAQLA | −75.43 | 0 | 0 | 0 | 0 | 1 | 3 |
| YEEFHAELA | −74.19 | 0 | 0 | 0 | 0 | 0 | 3 |
| YEELHAKLA | −73.61 | 0 | 0 | 0 | 0 | 2 | 3 |
| YQEFHARLA | −73.33 | 0 | 0 | 2 | 0 | 0 | 2 |
| YEELHAELA | −72.93 | 0 | 0 | 0 | 0 | 2 | 3 |
| YKEFHARLA | −72.81 | 0 | 0 | 2 | 0 | 0 | 2 |
| YREFHAQLA | −72.22 | 0 | 1 | 2 | 0 | 1 | 3 |
| YQEFHAQLA | −71.18 | 0 | 0 | 0 | 0 | 1 | 3 |
| YREFHAELA | −71.02 | 0 | 0 | 0 | 0 | 0 | 3 |
| YKEFHAQLA | −70.79 | 0 | 0 | 0 | 0 | 1 | 3 |
| YQEFHAELA | −69.99 | 0 | 0 | 0 | 0 | 0 | 3 |
| YRELHAKLA | −69.94 | 0 | 1 | 2 | 0 | 2 | 3 |
| YRELHAELA | −69.77 | 0 | 1 | 1 | 0 | 2 | 3 |
| YKEFHAELA | −69.60 | 0 | 0 | 0 | 0 | 0 | 3 |
| YQELHAKLA | −69.31 | 0 | 0 | 0 | 0 | 2 | 3 |
| YQELHAELA | −68.73 | 0 | 0 | 0 | 0 | 2 | 3 |
| YKELHAKLA | −68.47 | 0 | 0 | 0 | 0 | 2 | 3 |
| YKELHAELA | −68.35 | 0 | 0 | 0 | 0 | 2 | 3 |
| YEELHQRLA | −68.15 | 0 | 0 | 0 | 0 | 0 | 2 |
| YEEFHQQLA | −66.52 | 0 | 0 | 0 | 0 | 1 | 1 |
| LEELHARLA | −65.86 | 0 | 0 | 0 | 0 | 0 | 1 |
| YEEFHQELA | −65.49 | 0 | 0 | 0 | 0 | 0 | 1 |
| YEELHQQLA | −65.37 | 0 | 0 | 0 | 0 | 0 | 2 |
| LEQFHAQLA | −65.33 | 0 | 0 | 0 | 0 | 1 | 1 |
| LEEFHAQLA | −64.87 | 0 | 0 | 0 | 0 | 1 | 1 |
| LEQFHAELA | −64.85 | 0 | 0 | 0 | 0 | 0 | 1 |
| LEQFHAKLA | −64.45 | 0 | 0 | 0 | 0 | 0 | 2 |
| YEELHQELA | −64.23 | 0 | 0 | 0 | 0 | 0 | 1 |
| LEEFHAKLA | −64.04 | 0 | 0 | 0 | 0 | 0 | 2 |
| YQELHQRLA | −63.85 | 0 | 0 | 0 | 0 | 0 | 2 |
| YEEFHQKLA | −63.82 | 0 | 0 | 0 | 0 | 0 | 2 |
| LEEFHAELA | −63.63 | 0 | 0 | 0 | 0 | 0 | 1 |

### Example 4

### Experimental Confirmation of Predicted CNTF Epitopes

The baseline immunogenicity for Axokine® was established with an in vitro T-cell assay that measures secretion of cytokines (preference given to tests with IFN-g) by CD4+ T cells co-cultured with antigen presenting cells (APC). See Barbosa, M. D. F. S. et al. 2003. Testing MHC-binding epitopes: In vitro vaccination (IVV). Poster presented at the 90th Meeting of the American Society of Immunologists. Denver, Colo., May 6-10.

Several parameters were optimized for maximum uptake and processing of the proteins by APCs. A sensitive EliSPOT assay was used to detect T-cell activation as a measure of IFN-g produced. Blood from immunized patients are used for the tests or naïve T cells were primed in vitro with multiple rounds of exposure to APCs (Xencor “in vitro vaccination technology”).

Based on the results identified in Examples 1-3, a library of ENTF variant proteins were generated by cloning and expression in mammalian cells, using methodology that is well known in the art.

The ENTF variants were tested with a functional assay and the variants showing a specific activity were selected. The assays used include:

Proliferation assay using Ba/f3 cells transfected with the CNTF a-receptor (CNTFR), leukemia inhibitor factor receptor-b (LIFR), and the signal transducer gpl 306.

Dose-dependent proliferation of human TF-1 cells.

Binding assays have also been described, e.g., an assay that tests the ability of CNTF variants to compete with biotinylated CNTF for binding to the extracellular domain of myc-tagged CNTFR-a4.

Following selection of the predicted less immunogenic variants that retained function, the regions with reduced immunogenicity were tested in a T-cell assay, as described in U.S. Ser. No. 60/467,189, filed Apr. 30, 2003, hereby incorporated by reference in its entirety.

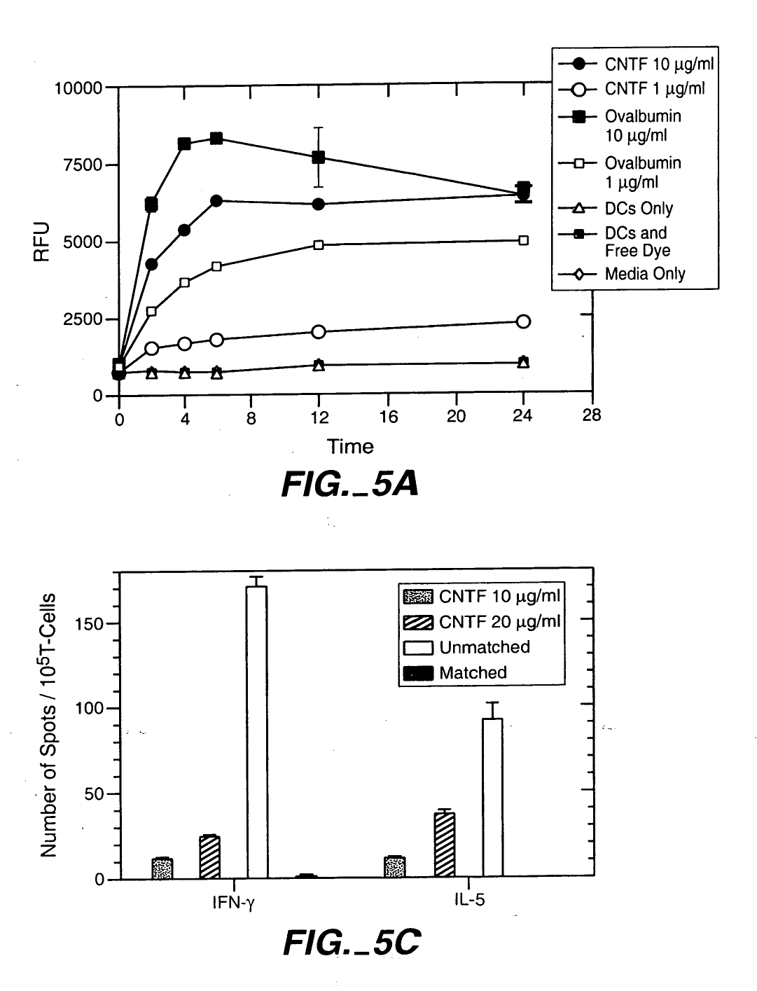

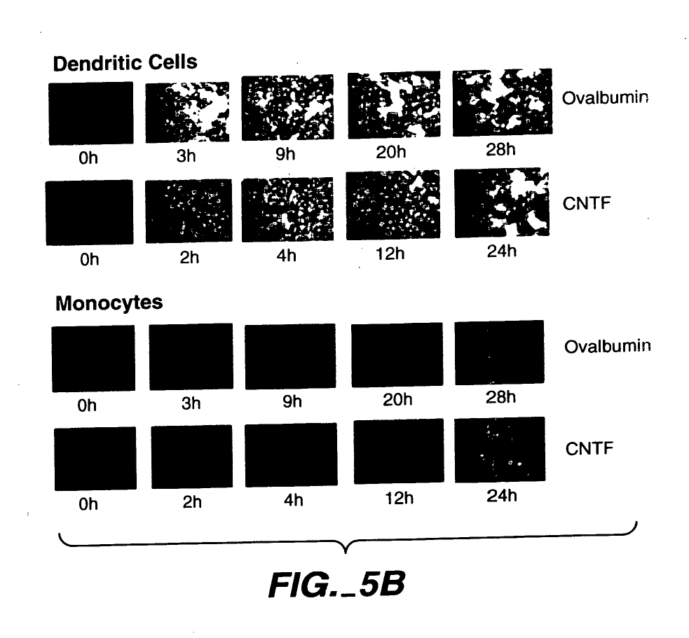

The results of the T-cell assay are show in FIGS. 5A and 5B. FIG. 5A shows that receptor mediated endocytosis may be the major mechanism of CNTF uptake by dendritic cells. FIG. 5B shows that T-cell proliferation was observed with a DRB1 allele representing approximately 30% of the American population.

All references cited herein, including patents, patent applications (provisional, utility and PCT), and publications are incorporated by reference in their entirety. The following patents and applications are incorporated by reference in their entirety:

| | |
|---|---|
| 4,997,929 | Synergen |
| 5,011,914 | CIP of 4,997,929. |
| 5,141,856 | CIP of 5,011,914. Synergen |
| 5,349,056 | Regeneron |
| 5,141,856 | CIP from 4,997,929. Synergen |
| 5,593,857 | Scios |
| 5,780,600 | Another in the 4,997,929 family. now assignee is |
| | Amgen. |
| 5,846,935 | CIP of 5,349,056. Regeneron |
| 5,939,534 | Sumitomo Pharma. (also EP 0749980) |
| 6,410,510 | Treating patients with variant hCNTF Regeneron |
| 6,440,702 | Another in the 5,349,056 family, Regeneron |
| 6,472,178 | CIP from 5,846,935. Regeneron |
| EPA 0,946,189 | Instituto di Richerche di Biologia Molecolare |
| US 2002 0123462 | In 5,349,056 family. Regeneron |
| WO 93/02206 | Syntex/Synergen 1993. Related to FP 0596034. |
| WO 93/10233 | Instituto di Richerche di Biologia Molecolare |
| WO 98/01149 | Instituto di Richerche di Biologia Molecolare |
| WO 98/22128 | Instituto di Richerche di Biologia Molecolare |
| 98/41625 | Instituto di Richerche di Biologia Molecolare |
| 02/070698 | Merck GMBH |

Barbosa, M. D. F. S. et al. 2003. Testing MHC-binding epitopes: In vitro vaccination (IVV). Poster presented at the 90th Meeting of the American Society of Immunologists. Denver, Colo., May 6-10

Czerkinsky, C. C. et al. 1983. A solid-phase enzyme-linked immunospot (ELISPOT) assay for enumeration of specific antibody-secreting cells. J. Immunol. Meth. 65: 109-

Davis, S. et al. 1993. LIFR beta and gp130 as heterodimerizing signal transducers of the tripartite CNTF receptor. Science. 260: 1805-1808

Di Marco, A. et al. 1996. Identification of ciliary neutrophic factor (CNTF) residues essential for leukemia inhibitory factor receptor binding and generation of CNTF receptor agonists. Proc. Nat. Acad. Sci. U. S. A. 93: 9247-9252

Filikov, A. V. et al. 2002. Computational stabilization of human growth hormone. Protein Sci. 11: 1452-1461

Gearing, D. P. et al. 1994. Proliferative responses and binding properties of hematopoietic cells transfected with low-affinity receptors for leukemia inhibitory factor, oncostatin M, and ciliary neurotropic factor. Proc. Natl. Acad. Sci. 91: 1119-1123

Hayes, R. J. et al. Combining computational and experimental screening for rapid optimization of protein properties. Proc. Nat. Acad. Sci. U. S. A.

Kallen, K.-J. 1999. Receptor recognition sites of cytokines are organized as exchangeable modules. J. Biol. Chem. 274: 11859-11867

Lambert, et al. 2001. Ciliary neurotrophic factor activates leptin-like pathways and reduces body fat, without cachexia or rebound weight gain, even in leptin-resistant obesity. Proc. Nat. Acad. Sci. U.S.A. 98: 4652-4657

Luo, P. et al. 2002. Development of a cytokine analog with enhanced stability using computational ultrahigh throughput screening. Protein Sci. 11: 1218-1226

Masiakowski P. et al. 1991. Recombinant human and rat ciliary neurotrophic factors. Neurochem. 57: 1003-1012

Marshall, S. A. et al. 2003. Rational design and engineering of therapeutic proteins. Drug Discov. Today. 8: 212-221

McDonald, N. Q. et al. 1995. Crystal structure of dimeric human ciliary neurotrphic factor determined by MAD phasing. EMBO J. 14: 2689-2699

Negro, A. et. al. 1994. Establishment and characterization of a CHO cell line producing a secreted form of human ciliary neurotrophic factor: neuroprotective effects of the recombinant protein. Mol. Brain Res. 21: 124-132

Osborne, R. 2003. Regeneron plummets on early phase III results with axokine. BioWorld Today. 14:1-

Panayotatos, N. et al. Localization of functional receptor epitopes on the structure of ciliary neurotropic factor indicates a conserved, function-related epitope topography among helical cytokines. J. Biol. Chem. 270: 1407-14014

Schuster, B. et al. 2003. Signaling of human ciliary neutrophic factor (CNTF) revisited. J. Biol. Chem. 278: 9528-9535

Sleeman, M. W. et al. 2000. The ciliary neurotrophic factor and its receptor, CNTFRa. Pharmaceutical Acta Helvetiae. 74: 265-272

Tang, L. et al. 2003. Expression and characterization of recombinant canine IL-13 receptor a2 protein and its biological activity in vitro. Mol. Immunol. 39: 719-727

Winter, C. G. et al. 1995. A role for ciliary neurotrophic factor as an inducer of reactive gliosis, the glial response to central nervous system injury. Proc. Natl. Acad. Sci. 92: 5865-5869

1. A modified ciliary neurotrophic factor (CNTF) protein having reduced immunogenicity as compared to a protein selected from the group consisting of SEQ ID NO:1 and SEQ ID NO:2.

2. A modified CNTF protein according to claim 1, wherein said protein has at least one substitution in a position selected from the group consisting of positions 21, 22, 23, 24, 25, 26 27, 28 and 29.

3. A modified CNTF protein according to claim 1, wherein said protein has at least one substitution in a position selected from the group consisting of positions 27, 28, 29, 30, 31, 32, 33, 34, and 35.

4. A modified CNTF protein according to claim 1, wherein said protein has at least one substitution in a position selected from the group consisting of positions 77, 78, 79, 80, 81, 82, 83, 84 and 85.

5. A modified CNTF protein according to claim 1, wherein said protein has at least one substitution in a position selected from the group consisting of positions 80, 81, 82, 83, 84, 85, 86, 87 and 88.

6. A modified CNTF protein according to claim 1, wherein said protein has at least one substitution in a position selected from the group consisting of positions 176, 177, 178, 179, 180, 181, 182, 183 and 184.

7. A modified CNTF protein according to claim 1, wherein said modified CNTF protein has reduced ability to bind one or more human class II MHC molecules.

8. A modified CNTF protein according to claim 1, wherein said protein has a amino acid substitution at position Y80.

9. A modified CNTF protein according to claim 8, wherein substitution is a non-hydrophobic amino acid.

10. A modified CNTF protein according to claim 8, wherein said non-hydrophobic residue is selected from the group consisting of D, E, G, H, K, N, Q, R, S, and T.

11. A recombinant nucleic acid encoding a CNTF protein of claim 1.

12. An expression vector comprising the recombinant nucleic acid of claim 11.

13. A host cell comprising the recombinant nucleic acid of claim 11.

14. A host cell comprising the expression vector of claim 13.

15. A method of producing a modified CNTF protein comprising culturing the host cell of claim 14 under conditions suitable for expression of said nucleic acid.

16. A method according to claim 15 further comprising recovering said protein.

17. A pharmaceutical composition comprising a modified CNTF according to claim 1 and a pharmaceutical carrier.

18. A method for treating a CNTF related disorder comprising administering a modified CNTF protein to a patient in need of said treatment.

19. A method according to claim 18, wherein said CNTF related disorder is obesity.

20. A method according to claim 18, wherein said patient possesses a DRB1 allele.


SEQUENCE LISTING (165 sequences)

SEQ ID NO: 1 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 2 - protein, 185 aa, Artificial
  synthetic
MAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTG

SEQ ID NO: 3 - protein, 9 aa, Homo sapiens
LCSRSIWLA

SEQ ID NO: 4 - protein, 9 aa, Homo sapiens
IWLARKIRS

SEQ ID NO: 5 - protein, 9 aa, Homo sapiens
WLARKIRSD

SEQ ID NO: 6 - protein, 9 aa, Homo sapiens
LARKIRSDL

SEQ ID NO: 7 - protein, 9 aa, Homo sapiens
IRSDLTALT

SEQ ID NO: 8 - protein, 9 aa, Homo sapiens
YVKHQGLNK

SEQ ID NO: 9 - protein, 9 aa, Homo sapiens
LNKNINLDS

SEQ ID NO: 10 - protein, 9 aa, Homo sapiens
INLDSADGM

SEQ ID NO: 11 - protein, 9 aa, Homo sapiens
LQAYRTFHV

SEQ ID NO: 12 - protein, 9 aa, Homo sapiens
YRTFHVLLA

SEQ ID NO: 13 - protein, 9 aa, Homo sapiens
FHVLLARLL

SEQ ID NO: 14 - protein, 9 aa, Homo sapiens
VLLARLLED

SEQ ID NO: 15 - protein, 9 aa, Homo sapiens
LLLQVAAFA

SEQ ID NO: 16 - protein, 9 aa, Homo sapiens
LLQVAAFAY

SEQ ID NO: 17 - protein, 9 aa, Homo sapiens
YQIEELMIL

SEQ ID NO: 18 - protein, 9 aa, Homo sapiens
LMILLEYKI

SEQ ID NO: 19 - protein, 9 aa, Homo sapiens
LEYKIPRNE

SEQ ID NO: 20 - protein, 9 aa, Homo sapiens
YKIPRNEAD

SEQ ID NO: 21 - protein, 9 aa, Homo sapiens
LWGLKVLQE

SEQ ID NO: 22 - protein, 9 aa, Homo sapiens
WGLKVLQEL

SEQ ID NO: 23 - protein, 9 aa, Homo sapiens
LKVLQELSQ

SEQ ID NO: 24 - protein, 9 aa, Homo sapiens
LSQWTVRSI

SEQ ID NO: 25 - protein, 9 aa, Homo sapiens
WTVRSIHDL

SEQ ID NO: 26 - protein, 9 aa, Homo sapiens
VRSIHDLRF

SEQ ID NO: 27 - protein, 9 aa, Homo sapiens
LRFISSHQT

SEQ ID NO: 28 - protein, 9 aa, Homo sapiens
FISSHQTGI

SEQ ID NO: 29 - protein, 9 aa, Artificial
  synthetic
YEEFHQRLA

SEQ ID NO: 30 - protein, 9 aa, Artificial
  synthetic
YKEFHQRLA

SEQ ID NO: 31 - protein, 9 aa, Artificial
  synthetic
YQEFHQRLA

SEQ ID NO: 32 - protein, 9 aa, Artificial
  synthetic
LEEFHARLA

SEQ ID NO: 33 - protein, 9 aa, Artificial
  synthetic
LEEFHQRLA

SEQ ID NO: 34 - protein, 9 aa, Artificial
  synthetic
LEELHAELA

SEQ ID NO: 35 - protein, 9 aa, Artificial
  synthetic
LEELHAKLA

SEQ ID NO: 36 - protein, 9 aa, Artificial
  synthetic
LEQFHARLA

SEQ ID NO: 37 - protein, 9 aa, Artificial
  synthetic
LKEFHARLA

SEQ ID NO: 38 - protein, 9 aa, Artificial
  synthetic
LKEFHQRLA

SEQ ID NO: 39 - protein, 9 aa, Artificial
  synthetic
LKELHAELA

SEQ ID NO: 40 - protein, 9 aa, Artificial
  synthetic
LKELHAKLA

SEQ ID NO: 41 - protein, 9 aa, Artificial
  synthetic
LQEFHARLA

SEQ ID NO: 42 - protein, 9 aa, Artificial
  synthetic
LQEFHQRLA

SEQ ID NO: 43 - protein, 9 aa, Artificial
  synthetic
LQELHAELA

SEQ ID NO: 44 - protein, 9 aa, Artificial
  synthetic
LQELHAKLA

SEQ ID NO: 45 - protein, 9 aa, Artificial
  synthetic
YREFHQELA

SEQ ID NO: 46 - protein, 9 aa, Artificial
  synthetic
YREFHQQLA

SEQ ID NO: 47 - protein, 9 aa, Artificial
  synthetic
YRELHQELA

SEQ ID NO: 48 - protein, 9 aa, Artificial
  synthetic
YRELHQKLA

SEQ ID NO: 49 - protein, 9 aa, Artificial
  synthetic
YEEFHQELA

SEQ ID NO: 50 - protein, 9 aa, Artificial
  synthetic
YEEFHQQLA

SEQ ID NO: 51 - protein, 9 aa, Artificial
  synthetic
YEELHQELA

SEQ ID NO: 52 - protein, 9 aa, Artificial
  synthetic
YEELHQKLA

SEQ ID NO: 53 - protein, 9 aa, Artificial
  synthetic
YKEFHQELA

SEQ ID NO: 54 - protein, 9 aa, Artificial
  synthetic
YKEFHQQLA

SEQ ID NO: 55 - protein, 9 aa, Artificial
  synthetic
YKELHQELA

SEQ ID NO: 56 - protein, 9 aa, Artificial
  synthetic
YKELHQKLA

SEQ ID NO: 57 - protein, 9 aa, Artificial
  synthetic
YQEFHQELA

SEQ ID NO: 58 - protein, 9 aa, Artificial
  synthetic
YQEFHQQLA

SEQ ID NO: 59 - protein, 9 aa, Artificial
  synthetic
YQELHQELA

SEQ ID NO: 60 - protein, 9 aa, Artificial
  synthetic
YQELHQKLA

SEQ ID NO: 61 - protein, 9 aa, Artificial
  synthetic
LREFHAELA

SEQ ID NO: 62 - protein, 9 aa, Artificial
  synthetic
LREFHQELA

SEQ ID NO: 63 - protein, 9 aa, Artificial
  synthetic
LREFHQQLA

SEQ ID NO: 64 - protein, 9 aa, Artificial
  synthetic
LEEFHAELA

SEQ ID NO: 65 - protein, 9 aa, Artificial
  synthetic
LEEFHAQLA

SEQ ID NO: 66 - protein, 9 aa, Artificial
  synthetic
LEEFHQELA

SEQ ID NO: 67 - protein, 9 aa, Artificial
  synthetic
LEEFHQQLA

SEQ ID NO: 68 - protein, 9 aa, Artificial
  synthetic
LEELHAQLA

SEQ ID NO: 69 - protein, 9 aa, Artificial
  synthetic
LEELHARLA

SEQ ID NO: 70 - protein, 9 aa, Artificial
  synthetic
LEQFHAELA

SEQ ID NO: 71 - protein, 9 aa, Artificial
  synthetic
LEQFHAQLA

SEQ ID NO: 72 - protein, 9 aa, Artificial
  synthetic
LKEFHAELA

SEQ ID NO: 73 - protein, 9 aa, Artificial
  synthetic
LKEFHAQLA

SEQ ID NO: 74 - protein, 9 aa, Artificial
  synthetic
LKEFHQELA

SEQ ID NO: 75 - protein, 9 aa, Artificial
  synthetic
LKEFHQQLA

SEQ ID NO: 76 - protein, 9 aa, Artificial
  synthetic
LKELHAQLA

SEQ ID NO: 77 - protein, 9 aa, Artificial
  synthetic
LKELHARLA

SEQ ID NO: 78 - protein, 9 aa, Artificial
  synthetic
LKQFHAELA

SEQ ID NO: 79 - protein, 9 aa, Artificial
  synthetic
LQEFHAELA

SEQ ID NO: 80 - protein, 9 aa, Artificial
  synthetic
LQEFHAQLA

SEQ ID NO: 81 - protein, 9 aa, Artificial
  synthetic
LQEFHQELA

SEQ ID NO: 82 - protein, 9 aa, Artificial
  synthetic
LQEFHQQLA

SEQ ID NO: 83 - protein, 9 aa, Artificial
  synthetic
LQELHAQLA

SEQ ID NO: 84 - protein, 9 aa, Artificial
  synthetic
LQELHARLA

SEQ ID NO: 85 - protein, 9 aa, Artificial
  synthetic
LQQFHAELA

SEQ ID NO: 86 - protein, 9 aa, Artificial
  synthetic
YREFHQKLA

SEQ ID NO: 87 - protein, 9 aa, Artificial
  synthetic
YRELHQQLA

SEQ ID NO: 88 - protein, 9 aa, Artificial
  synthetic
YEEFHARLA

SEQ ID NO: 89 - protein, 9 aa, Artificial
  synthetic
YEEFHQKLA

SEQ ID NO: 90 - protein, 9 aa, Artificial
  synthetic
YEELHQQLA

SEQ ID NO: 91 - protein, 9 aa, Artificial
  synthetic
YEELHQRLA

SEQ ID NO: 92 - protein, 9 aa, Artificial
  synthetic
YKEFHQKLA

SEQ ID NO: 93 - protein, 9 aa, Artificial
  synthetic
YKELHQQLA

SEQ ID NO: 94 - protein, 9 aa, Artificial
  synthetic
YKELHQRLA

SEQ ID NO: 95 - protein, 9 aa, Artificial
  synthetic
YQEFHQKLA

SEQ ID NO: 96 - protein, 9 aa, Artificial
  synthetic
YQELHQQLA

SEQ ID NO: 97 - protein, 9 aa, Artificial
  synthetic
YQELHQRLA

SEQ ID NO: 98 - protein, 9 aa, Artificial
  synthetic
LREFHVELA

SEQ ID NO: 99 - protein, 9 aa, Artificial
  synthetic
LREFHAKLA

SEQ ID NO: 100 - protein, 9 aa, Artificial
  synthetic
LREFHQKLA

SEQ ID NO: 101 - protein, 9 aa, Artificial
  synthetic
LRELHVELA

SEQ ID NO: 102 - protein, 9 aa, Artificial
  synthetic
LEAFHARLA

SEQ ID NO: 103 - protein, 9 aa, Artificial
  synthetic
LEEFHVELA

SEQ ID NO: 104 - protein, 9 aa, Artificial
  synthetic
LEEFHAKLA

SEQ ID NO: 105 - protein, 9 aa, Artificial
  synthetic
LEEFHQKLA

SEQ ID NO: 106 - protein, 9 aa, Artificial
  synthetic
LEELHVELA

SEQ ID NO: 107 - protein, 9 aa, Artificial
  synthetic
LEQFHVELA

SEQ ID NO: 108 - protein, 9 aa, Artificial
  synthetic
LEQFHAKLA

SEQ ID NO: 109 - protein, 9 aa, Artificial
  synthetic
LKEFHVELA

SEQ ID NO: 110 - protein, 9 aa, Artificial
  synthetic
LKEFHAKLA

SEQ ID NO: 111 - protein, 9 aa, Artificial
  synthetic
LKEFHQKLA

SEQ ID NO: 112 - protein, 9 aa, Artificial
  synthetic
LKELHVELA

SEQ ID NO: 113 - protein, 9 aa, Artificial
  synthetic
LKQFHAKLA

SEQ ID NO: 114 - protein, 9 aa, Artificial
  synthetic
LQEFHVELA

SEQ ID NO: 115 - protein, 9 aa, Artificial
  synthetic
LQEFHAKLA

SEQ ID NO: 116 - protein, 9 aa, Artificial
  synthetic
LQEFHQKLA

SEQ ID NO: 117 - protein, 9 aa, Artificial
  synthetic
LQELHVELA

SEQ ID NO: 118 - protein, 9 aa, Artificial
  synthetic
LQQFHAKLA

SEQ ID NO: 119 - protein, 9 aa, Artificial
  synthetic
YREFHAELA

SEQ ID NO: 120 - protein, 9 aa, Artificial
  synthetic
YEEFHAELA

SEQ ID NO: 121 - protein, 9 aa, Artificial
  synthetic
YEEFHAQLA

SEQ ID NO: 122 - protein, 9 aa, Artificial
  synthetic
YEELHAELA

SEQ ID NO: 123 - protein, 9 aa, Artificial
  synthetic
YEELHAKLA

SEQ ID NO: 124 - protein, 9 aa, Artificial
  synthetic
YKEFHAELA

SEQ ID NO: 125 - protein, 9 aa, Artificial
  synthetic
YKEFHAQLA

SEQ ID NO: 126 - protein, 9 aa, Artificial
  synthetic
YKELHAELA

SEQ ID NO: 127 - protein, 9 aa, Artificial
  synthetic
YKELHAKLA

SEQ ID NO: 128 - protein, 9 aa, Artificial
  synthetic
YQEFHAELA

SEQ ID NO: 129 - protein, 9 aa, Artificial
  synthetic
YQEFHAQLA

SEQ ID NO: 130 - protein, 9 aa, Artificial
  synthetic
YQELHAELA

SEQ ID NO: 131 - protein, 9 aa, Artificial
  synthetic
YQELHAKLA

SEQ ID NO: 132 - protein, 9 aa, Artificial
  synthetic
LRELHLELA

SEQ ID NO: 133 - protein, 9 aa, Artificial
  synthetic
LRELHQELA

SEQ ID NO: 134 - protein, 9 aa, Artificial
  synthetic
LRELHQKLA

SEQ ID NO: 135 - protein, 9 aa, Artificial
  synthetic
LEAFHAELA

SEQ ID NO: 136 - protein, 9 aa, Artificial
  synthetic
LEAFHAQLA

SEQ ID NO: 137 - protein, 9 aa, Artificial
  synthetic
LEELHLELA

SEQ ID NO: 138 - protein, 9 aa, Artificial
  synthetic
LEELHQELA

SEQ ID NO: 139 - protein, 9 aa, Artificial
  synthetic
LEELHQKLA

SEQ ID NO: 140 - protein, 9 aa, Artificial
  synthetic
LKAFHAELA

SEQ ID NO: 141 - protein, 9 aa, Artificial
  synthetic
LKELHLELA

SEQ ID NO: 142 - protein, 9 aa, Artificial
  synthetic
LKELHQELA

SEQ ID NO: 143 - protein, 9 aa, Artificial
  synthetic
LKELHQKLA

SEQ ID NO: 144 - protein, 9 aa, Artificial
  synthetic
LQAFHAELA

SEQ ID NO: 145 - protein, 9 aa, Artificial
  synthetic
LQELHLELA

SEQ ID NO: 146 - protein, 9 aa, Artificial
  synthetic
LQELHQELA

SEQ ID NO: 147 - protein, 9 aa, Artificial
  synthetic
LQELHQKLA

SEQ ID NO: 148 - protein, 9 aa, Artificial
  synthetic
LRELHAELA

SEQ ID NO: 149 - protein, 9 aa, Artificial
  synthetic
LRELHAKLA

SEQ ID NO: 150 - protein, 9 aa, Artificial
  synthetic
LREFHAQLA

SEQ ID NO: 151 - protein, 9 aa, Artificial
  synthetic
LKQFHAQLA

SEQ ID NO: 152 - protein, 9 aa, Artificial
  synthetic
LQQFHAQLA

SEQ ID NO: 153 - protein, 9 aa, Artificial
  synthetic
YKEFHARLA

SEQ ID NO: 154 - protein, 9 aa, Artificial
  synthetic
YQEFHARLA

SEQ ID NO: 155 - protein, 9 aa, Artificial
  synthetic
LKQFHVELA

SEQ ID NO: 156 - protein, 9 aa, Artificial
  synthetic
LQQFHVELA

SEQ ID NO: 157 - protein, 9 aa, Artificial
  synthetic
YEQFHARLA

SEQ ID NO: 158 - protein, 9 aa, Artificial
  synthetic
LKAFHAQLA

SEQ ID NO: 159 - protein, 9 aa, Artificial
  synthetic
LQAFHAQLA

SEQ ID NO: 160 - protein, 9 aa, Artificial
  synthetic
LREFHQRLA

SEQ ID NO: 161 - protein, 9 aa, Artificial
  synthetic
YRELHAELA

SEQ ID NO: 162 - protein, 9 aa, Artificial
  synthetic
LRELHAQLA

SEQ ID NO: 163 - protein, 9 aa, Artificial
  synthetic
YREFHAQLA

SEQ ID NO: 164 - protein, 9 aa, Artificial
  synthetic
YRELHAKLA

SEQ ID NO: 165 - protein, 9 aa, Artificial
  synthetic
YRELHQRLA